(19) United States
(12) Patent Application Publication
(10) Pub. No.: US 2025/0057885 A1
(43) Pub. Date: Feb. 20, 2025

(54) PLATELET-DERIVED EXTRACELLULAR VESSICLES FOR TREATMENT OF CARDIOGENIC SHOCK AND SEPSIS

(71) Applicant: Mitrix Bio, Inc., Pleasanton, CA (US)

(72) Inventor: Thomas B. Benson, Pleasanton, CA (US)

(21) Appl. No.: 18/936,814

(22) Filed: Nov. 4, 2024

Related U.S. Application Data
(63) Continuation of application No. PCT/US2023/021259, filed on May 5, 2023.
(60) Provisional application No. 63/364,409, filed on May 9, 2022.

(51) Int. Cl. A61K 35/19; A61K 9/50; A61P 9/02; A61P 37/06

(52) U.S. Cl. CPC A61K 35/19; A61K 9/5068; A61P 9/02; A61P 37/06

(57) ABSTRACT

A subject can be treated for cardiogenic shock and/or sepsis, or their symptoms using platelet-derived extracellular vesicles (PEVs) or mitlets that include PEVS. The PEVs include mitochondria. The PEVs can be collected by obtaining blood from one or more donors, adding an anticoagulant and a buffer to the blood to form a mix, separating the mix into supernatant and platelet rich plasma (PRP), collecting the PRP and stimulating the collected PRP, thereby expelling extracellular vesicles from platelets in the PRP, and collecting the extracellular vesicles as the PEVs. PEVs can also be isolated from source cells grown in a bioreactor and suspended in a buffer to preserve the PEVs.

### INCORPORATION BY REFERENCE TO ANY PRIORITY APPLICATIONS

This application is a continuation of PCT Application Serial No. PCT/US2023/021259, filed May 5, 2023, which claims priority to U.S. Provisional Application Ser. No. 63/364,409, filed May 9, 2022. Each of the aforementioned applications is incorporated herein by reference in its entirety.

### BACKGROUND

### Field

The presently disclosed and claimed inventions relate generally to a methodology of treating cardiogenic shock and/or sepsis indications, and more particularly to such methodology that involves delivery of mitlets, which include platelet-derived extracellular vesicles (PEVs) that include mitochondria.

### Description of the Related Art

Mitochondria are membrane-limited subcellular organelles that contain their own DNA (mtDNA) and their own machinery for synthesizing RNA and proteins. They are found in nearly all eukaryotic cells and vary in number and location depending on the cell type.

Mitochondria perform numerous essential tasks in the eukaryotic cell such as pyruvate oxidation, the Krebs cycle and metabolism of amino acids, fatty acids, and steroids. The primary function of mitochondria is the generation of energy as adenosine triphosphate (ATP) by means of the electron-transport chain and the oxidative-phosphorylation system (the “respiratory chain”). Additional processes in which mitochondria are involved include heat production, storage of calcium ions, calcium signaling, programmed cell death (apoptosis) and cellular proliferation. It has been disclosed that mitochondria have a role in cell regulatory and signaling events (e.g., regulation of Ca²⁺ fluxes, oxidative stress and energy-related signaling among others).

One manifestation of cardiovascular disease is cardiogenic shock, which occurs in approximately 5-8% of ST-elevation myocardial infarction (STEMI) and 2-3% of non-STEMI cases. The incidence trends of cardiogenic shock from acute myocardial infarction are shown in FIG. 1. This translates to about 50,000 cases of cardiogenic shock per year in the United States, with a mortality rate in excess of 40%, which makes cardiogenic shock one of the leading causes of death in patients with acute myocardial infarction.

The pathophysiology of cardiogenic shock includes acute myocardial ischemia, which leads to myocardial dysfunction. A potentially catastrophic spiral of reduced cardiac output and low blood pressure may manifest themselves from the myocardial dysfunction, which perpetuates further coronary ischemia and impairment of the contractility of cardiac tissue. These processes, when added to infarct-induced tissue injury and to genetic and environmental risk factors, may trigger a cytokine storm (i.e., a systemic inflammatory response syndrome (SIRS)) as shown in FIG. 2. A cytokine storm is characterized by the elevated activation of inflammatory signaling pathways, which leads to a large release of pro-inflammatory cytokines, which may lead to sepsis. As shown in FIG. 2, a cytokine storm may be triggered by major tissue injury from an acute myocardial infarction associated with cardiogenic shock, which is a self-perpetuating cycle that leads to global hypoperfusion, which progresses to multiple organ failure, progressive cardiac dysfunction, and then death in many cases.

Additionally, 5-7% of patients having cardiogenic shock also develop sepsis. Cardiogenic shock patients with concomitant sepsis are at an elevated risk for adverse outcomes. The global hypoperfusion triggered by the cytokine storm and the increase production of nitric oxide (NO) may lead to vascular endothelial injury and circulatory organ dysfunction. Overproduction of NO leads to persistent vasodilation. Olwal et al. “Parallels in Sepsis and COVID-19 Conditions: Implications for Managing Severe COVID-19.” Front Immunol. 2021; 12:602848. It is also believed that vascular endothelial injury leads to leaky vessels, which may provide an entryway into the circulatory system for opportunistic pathogens, such as bacteria, viruses, fungi, or parasites. The mortality rate of cardiogenic shock patients who develop sepsis is at an excess of 40%.

Additionally, the pneumonia condition called coronavirus disease 2019 (COVID-19) caused by severe acute respiratory coronavirus 2 (SARS-CoV-2) features many pathophysiological and clinical parallels to sepsis. As shown in FIG. 3, severe cases of COVID-19 are correlated with high levels of pro-inflammatory cytokines measured in the bloodstream, these cytokines include, but are not limited to, interleukin (IL) 6 (IL-6), IL-10, tumor necrosis factor α (TNFα), colony-stimulating factor (CSF), and interferon-inducible protein 10 (IP10). As in sepsis from cardiogenic shock patients, sepsis caused by COVID-19 may result in sepsis and tissue damage, which can lead to multiple organ failure. Furthermore, Long COVID-19 conditions (COVID-19 symptoms, in individuals having probable or confirmed SARS-CoV-2 infection, persisting for at least 2 months following 3 months from onset of COVID-19) may make a patient more susceptible to cardiogenic shock, and sepsis thereafter.

Some have approached the problem of treating cardiogenic shock by emergency revascularization. Early aggressive therapy is warranted because about 80% of the deaths from cardiogenic shock occur within 30 days as shown in FIG. 4. TRIUMPH Investigators et al. “Effect of tilarginine acetate in patients with acute myocardial infarction and cardiogenic shock: the TRIUMPH randomized controlled trial.” JAMA. 2007; 297(15):1657-1666. Also shown, survivors at 90 d have quite a favorable prognosis over the next several months. The data from the TRIUMPH study suggests that if a successful aggressive early therapy can be developed, it would have a major impact on longer-term outcomes for patients with cardiogenic shock and potentially COVID-19. The technique of mitochondrial transfusion—gathering mitochondria from an outside source and transfusing into the body—has recently been developed by a number of major universities. While mitochondrial transfusion is generally less invasive than other approaches, finding a source of mitochondria and preparing the mitochondria for transfusion for treatment of the conditions described above has long been a challenge.

### SUMMARY OF THE INVENTION

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

In one aspect, the disclosed technology provides a method of treatment for a condition, or symptoms thereof, in a subject having the condition, comprising obtaining mitlets including platelet-derived extracellular vesicles (PEVs) that include mitochondria, wherein the PEVs are collected by: obtaining blood from one or more donors; adding an anticoagulant and a buffer to the blood to form a mix; separating the mix into supernatant and platelet rich plasma (PRP); collecting the PRP; stimulating the collected PRP, thereby expelling extracellular vesicles from platelets in the PRP; and collecting the extracellular vesicles as the PEVs; and administering an effective amount of the mitlets into the subject, thereby treating the condition, or the symptoms thereof. In some embodiments the PEVs have been collected at a different site than a site where the treatment is carried out.

In some embodiments, the condition comprises cardiogenic shock. In some embodiments, wherein the condition comprises sepsis. In some embodiments, the condition comprises a disease caused by a virus. In some embodiments, the virus comprises a coronavirus. In some embodiments, the coronavirus comprises severe acute respiratory coronavirus 2 (SARS-CoV-2). In some embodiments, the disease is coronavirus disease 2019 (COVID-19). In some embodiments, the condition comprises cardiogenic shock, sepsis, and a disease caused by a virus. In some embodiments, the disease is COVID-19. In some embodiments, the disease is Long COVID-19. In some embodiments, the COVID-19 precedes the cardiogenic shock, and the cardiogenic shock precedes the sepsis.

In some embodiments, the administering step comprises injecting the effective amount of mitlets into the subject to treat the condition. In some embodiments, the collected PRP is stimulated with immune complexes in presence of Ca2+. In some embodiments, the immune complexes comprise heat-aggregated IgG. In some embodiments, the collected PRP is stimulated by freeze-thaw cycles. In some embodiments, concentration of the heat-aggregated IgG is about 0.1 mg/mL to about 2.5 mg/mL, and wherein concentration of the Ca2+ is about 1 mM to about 25 mM. In some embodiments, the anticoagulant is anticoagulant citrate dextrose (ACD). In some embodiments, the buffer is Tyrode's buffer at about pH 6 to about pH 7. In some embodiments, the separating step is conducted by centrifuge. In some embodiments, the blood has been stored for four or more days. In some embodiments, the blood has been stored for up to one year.

In some embodiments, during and/or after the administering the mitlets into the subject, the mitlets contact at least one cell of the subject. In some embodiments, the mitlets are internalized into the cell after the mitlets contact the cell. In some embodiments, the effective amount corresponds to an amount of the internalized mitlets, which ranges from about 3 mitlets/cell to about 100 mitlets/cell. In some embodiments, the mitlets are frozen while stored. In some embodiments, the frozen mitlets are stored in combination with a cryoprotectant. In some embodiments, the cryoprotectant is selected from the group consisting of a saccharide, an oligosaccharide, and a polysaccharide.

In a second aspect, the disclosed technology provides a method of treatment for a condition, or symptoms thereof, in a subject having the condition, comprising obtaining PEVs from a source, wherein the PEVs comprise mitochondria; suspending the PEVs in a buffer to preserve the PEVs; and administering an effective amount of the PEVs into the subject, thereby treating the condition, or the symptoms thereof. In some embodiments, the source comprises a cell can be a stem cell, such as a cell selected from the group consisting of: placental stem cells, umbilical cord stem cells, adipose tissue-derived stem cells and induced pluripotent stem cells. The cell can also be selected from the group consisting of hepatocytes, blood cells, stem cells, or any cells from a donor. In some embodiments, the source comprises a tissue selected from the group consisting of: liver, bone marrow, placenta, adipose tissue, or any tissues from a donor.

In some embodiments, the obtaining step comprises growing the source in a bioreactor; and isolating the PEVs from the source grown in the bioreactor. In some embodiments, the obtaining step further comprise coating the PEVs after isolating step. In some embodiments, the PEVs have been collected at a different site than a site where the treatment is carried out.

In some embodiments, the condition comprises cardiogenic shock. In some embodiments, the condition comprises sepsis. In some embodiments, the condition comprises a disease caused by a virus. In some embodiments, the virus comprises a coronavirus. In some embodiments, the coronavirus comprises SARS-CoV-2. In some embodiments, the disease is COVID-19. In some embodiments, the condition comprises cardiogenic shock, sepsis, and a disease caused by a virus. In some embodiments, the disease is COVID-19. In some embodiments, the COVID-19 precedes the cardiogenic shock, and the cardiogenic shock precedes the sepsis. In some embodiments, the administering step comprises injecting the effective amount of mitlets into the subject to treat the condition.

In some embodiments, the buffer comprises a cryoprotectant. In some embodiments, the cryoprotectant is selected from the group consisting of a saccharide, an oligosaccharide, and a polysaccharide. In some embodiments, the buffer comprises a hydrogel. In some embodiments, the hydrogel has temperature-dependent hydrophilicity and hydrophobicity.

In some embodiments, mitlets are provided, the mitlets including platelet-derived extracellular vesicles (PEVs) that include mitochondria for use in the treatment of cardiogenic shock and/or sepsis, or symptoms thereof, wherein the PEVs are collected by: obtaining blood from one or more donors; adding an anticoagulant and a buffer to the blood to form a mix; separating the mix into supernatant and platelet rich plasma (PRP); collecting the PRP; stimulating the collected PRP, thereby expelling extracellular vesicles from platelets in the PRP; and collecting the extracellular vesicles as the PEVs. In some embodiments, the PEVs have been collected at a different site than a site where the treatment is carried out. In some embodiments, the mitlets are used in the treatment of cardiogenic shock. In some embodiments, the mitlets are used in the treatment of sepsis. In some embodiments, the cardiogenic shock and/or sepsis is caused by a virus. In some embodiments, the virus comprises a coronavirus. In some embodiments, the coronavirus comprises severe acute respiratory coronavirus 2 (SARS-CoV-2). In some embodiments, use in simultaneous treatment of cardiogenic shock, sepsis, and a disease caused by a virus. In some embodiments, the disease is COVID-19.

In some embodiments, the collected PRP is stimulated with immune complexes in presence of Ca2+. In some embodiments, the immune complexes comprise heat-aggregated IgG. In some embodiments, the collected PRP is stimulated by freeze-thaw cycles. In some embodiments, a concentration of the heat-aggregated IgG is about 0.1 mg/mL to about 2.5 mg/mL, and wherein concentration of the Ca2+ is about 1 mM to about 25 mM. In some embodiments, the anticoagulant is anticoagulant citrate dextrose (ACD). In some embodiments, the buffer is Tyrode's buffer at about pH 6 to about pH 7. In some embodiments, the separating step is conducted by centrifuge.

In some embodiments, the blood has been stored for four or more days. In some embodiments, the blood has been stored for up to one year. In some embodiments, an effective amount of the mitlets ranges from about 3 mitlets/cell to about 100 mitlets/cell. In some embodiments, the mitlets are frozen while stored. In some embodiments, the frozen mitlets are stored in combination with a cryoprotectant. In some embodiments, the cryoprotectant is selected from the group consisting of a saccharide, an oligosaccharide, and a polysaccharide.

In some embodiments, PEVs are provided, the PEVs comprising mitochondria for use in the treatment of cardiogenic shock and/or sepsis, or symptoms thereof, wherein the PEVs are suspended in a buffer to preserve the PEVs and are isolated from source cells grown in a bioreactor, wherein the source cells obtained are selected from the group consisting of: placental stem cells, umbilical cord stem cells, adipose tissue-derived stem cells; hepatocytes, blood cells, bone marrow, and induced pluripotent stem cells. In some embodiments, the PEVs are coated. In some embodiments, an effective amount of the PEVs are administered into a subject as shown. In some embodiments, the PEVs are used in the treatment of cardiogenic shock. In some embodiments, the PEVs are used in the treatment of sepsis. In some embodiments, the cardiogenic shock and/or sepsis is caused by a virus. In some embodiments, the virus includes a coronavirus. In some embodiments, the coronavirus includes severe acute respiratory coronavirus 2 (SARS-CoV-2). In some embodiments, the PEVs are used in simultaneous treatment of cardiogenic shock, sepsis, and a disease caused by a virus.

In some embodiments, the disease is COVID-19. In some embodiments, the buffer comprises a cryoprotectant. In some embodiments, the cryoprotectant is selected from the group consisting of a saccharide, an oligosaccharide, and a polysaccharide. In some embodiments, the buffer comprises a hydrogel. In some embodiments, the hydrogel has temperature-dependent hydrophilicity and hydrophobicity.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows incidence trends of cardiogenic shock in acute myocardial infarction.

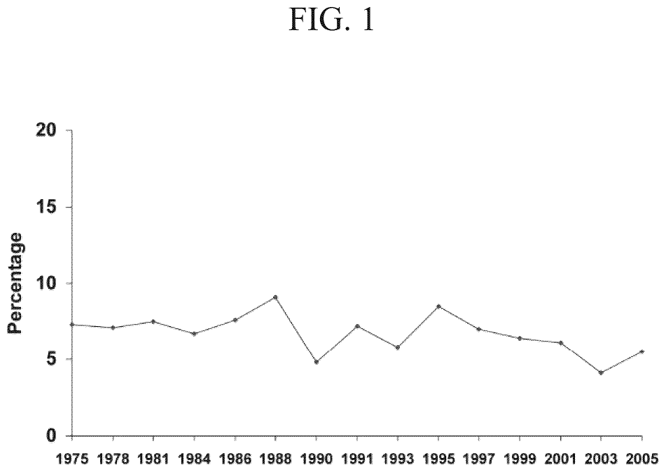

FIG. 2 depicts a pathway diagram of cardiogenic shock from an acute myocardial infarction that leads to a cytokine storm.

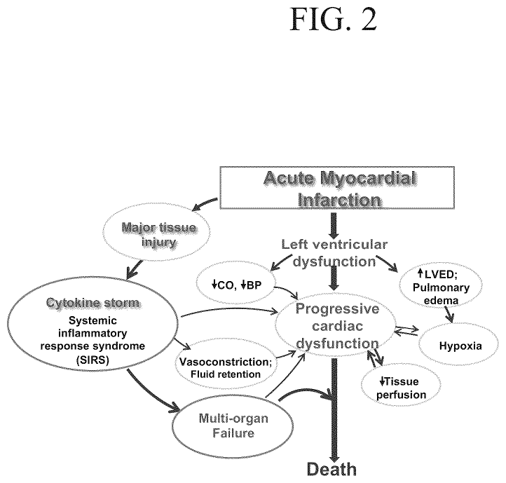

FIG. 3 depicts a pathway diagram of a cytokine storm in patient suffering from COVID-19.

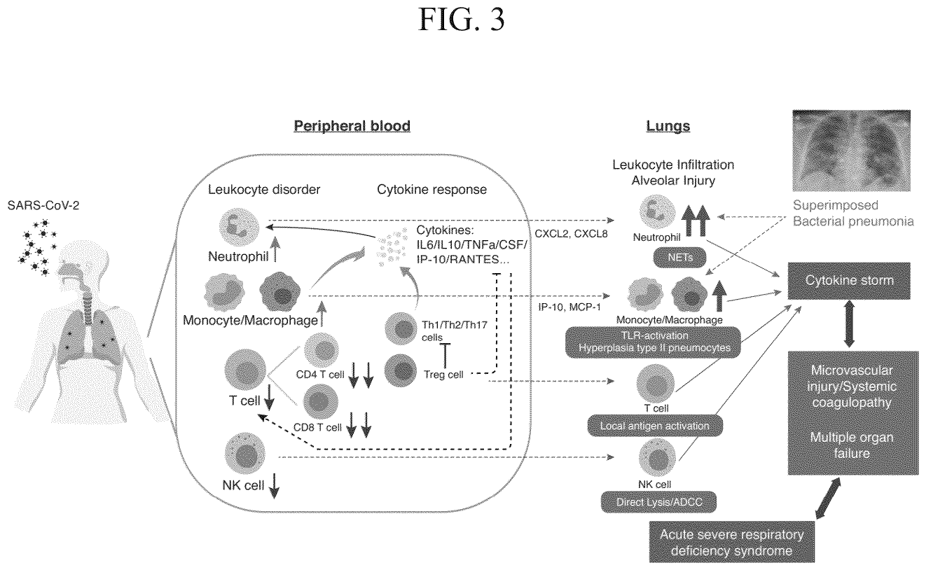

FIG. 4 shows the mortality rate from cardiogenic shock following acute myocardial infarction.

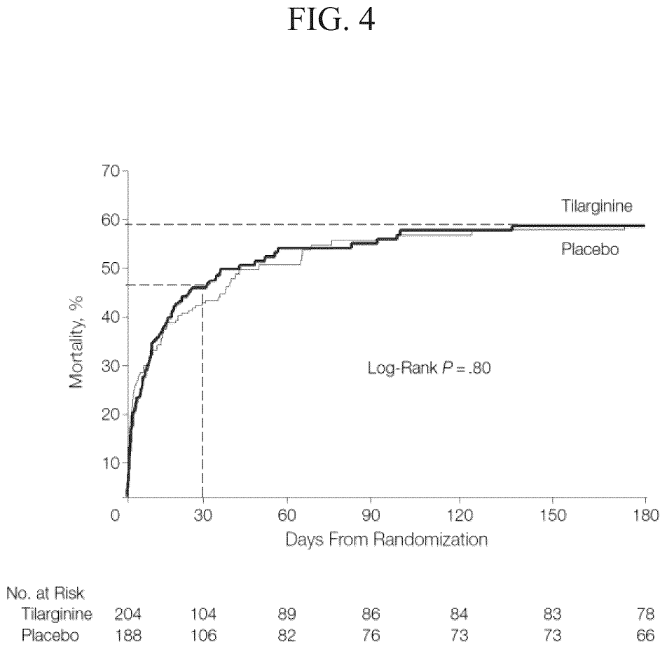

FIG. 5 is a schematic diagram showing a method of obtaining mitlets according to some embodiments.

FIG. 6 is a schematic diagram showing a method of obtaining PEVs according to some embodiments.

FIG. 7 is schematic diagram showing a method of obtaining mitlets according to some embodiments.

FIG. 8 is a schematic diagram showing a method of obtaining PEVs according to some embodiments.

FIG. 9 is a dot plot that represents PEV populations where the PEVs, labeled with DsRed, are represented as approximately 40% of the total CD41+PEVs.

FIGS. 10A and 10B are fluorescent images of retinal pigmented epithelium cells (RPEC) grown in culture showing uptake of PEVs into RPECs; FIG. 10A shown at 20× and FIG. 10B shown at 40×.

FIG. 11 are fluorescent images that show uptake and internalization of PEVs into cultured RPECs.

FIG. 12 is a fluorescent image showing internalized PEV-delivered mitochondria in cultured RPECs.

FIGS. 13A and 13B illustrate the uptake of PEVs of various effective amounts into different cell types, wherein FIG. 13A illustrates the uptake of PEVs into RPECs and FIG. 13B illustrates the uptake of PEVs into brain endothelial cells (bEND).

FIGS. 14A-14D show the oxygen consumption rate (OCR) at various stages of oxidative phosphorylation in the mitochondria of the RPECs where at least some of the mitochondria were transfused into the RPECs from PEVs at various effective amounts.

FIG. 15 are fluorescent images showing internalization of PEVs into cells of the bone marrow and spleen in vivo.

FIG. 16 shows a flowchart of the study design that evaluates the efficacy of mitlet treatment.

FIG. 17 shows the inflammatory response between randomized subjects receiving mitlet treatments and those that receive placebo.

FIG. 18 shows reduction of mortality in randomized subjects who received the mitlet treatment versus a TRIUMPH randomized control trial.

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Mitochondrial dysfunction is an underlying factor in multiple diseases including cardiovascular disease, cancer, Alzheimer's, diabetes, vision loss, and frailty. In some aspects of the present invention, mitochondria are transplanted to treat these and other diseases and conditions. Finding a source of mitochondria to transplant is a challenge. Just like any donated organ, mitochondria from young healthy donors are in short supply. Some diseases or injuries might be cured by autologous mitochondria—removed from a leg muscle in one's own body for example—however, for many other diseases, the “patients” have poor quality mitochondria due to age or mutation to mitochondrial DNA (mtDNA). For these patients, donated mitochondria are a preferred solution. In addition, freshly fully isolated mitochondria die quickly within minutes of isolation and may also provoke immune reactions when put naked into the bloodstream, reducing their effectiveness as a therapy. Therefore, it would be convenient to find an easy and readily available source of donation-ready mitochondria, which at the same time, are encased in some sort of coating, vesicle, or vehicle suspension (or any combination thereof) that protect them from the immune system.

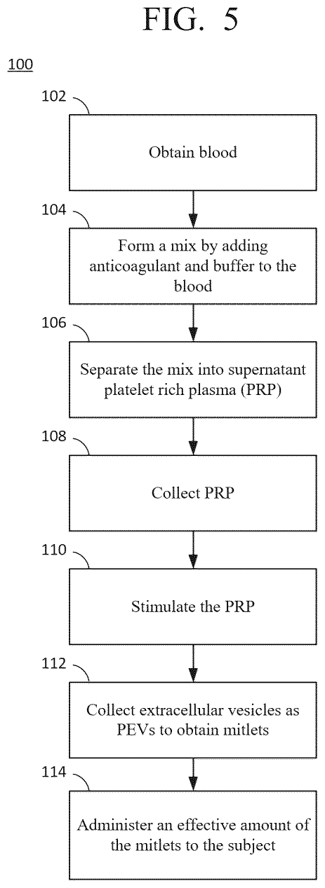

As shown in FIG. 5. Some embodiments relate to a method 100 of extracting platelet-derived mitochondria-containing extracellular vesicles (PEVs). In some embodiments, the method 100 includes 1) obtaining blood from donors in step 102, 2) adding anticoagulant and a buffer to the blood to form a mix in step 104, 3) separating the mix into supernatant and platelet rich plasma (PRP) in step 106, 4) collecting platelet rich plasma (PRP) in step 108, 5) stimulating the collected platelets in step 110, and collecting the PEVs in step 112. In some embodiments, mitlets comprise the PEVs.

A platelet from human blood contains 4-5 mitochondria on average that are expelled in extracellular vesicles when platelets are activated. The platelet-derived mitochondria-containing extracellular vesicles are referred to PEVs herein. These PEVs are usually larger (>400 nM), and less well-known than other platelet extracts or lysates (30-100 nM), however other sizes may also apply.

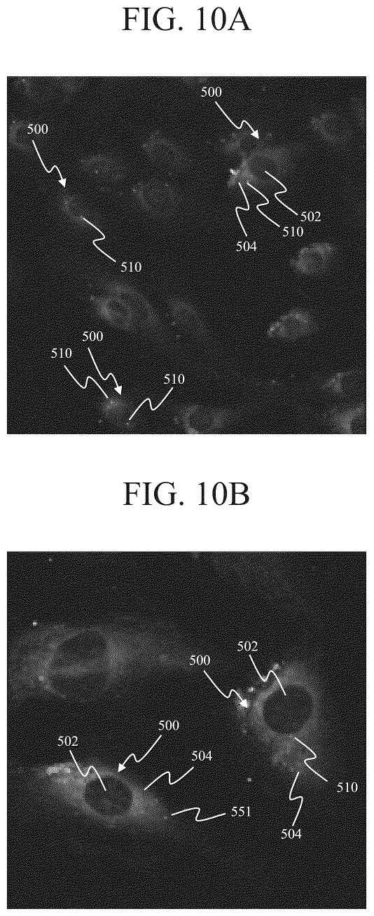

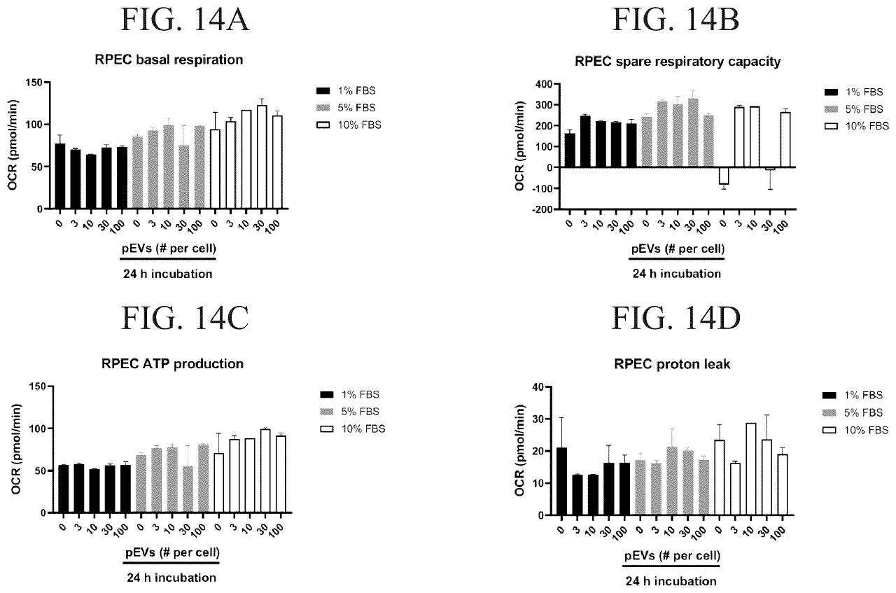

PEVs have been shown to donate their mitochondria to cells nearby (shown in FIGS. 10A-B, 11, and 12), which can increase the respiratory activity of the cells that absorb them as shown in FIGS. 14A-14D, thus regenerating tissue and curing several diseases of aging. PEVs have several advantages for fast commercialization: notably, they can be extracted from donated platelets that have “expired” and must be thrown away; they represent another good medically-valid use for platelets which otherwise might go to waste; they could be collected at most blood banks, who already have all the needed skilled personnel, clean handling practices, and equipment needed, and are already in close proximity to hospitals, thus making PEV product potentially available to world-wide use extremely soon. PEVs are a variety of platelet transfusion and therefore are more likely to be embraced and tested by medical professionals who are already familiar with blood transfusion therapies. Furthermore, PEVs can be prepared for localized transfusion into various internal anatomical regions to treat various clinical disorders using delivery devices already on the market.

In some embodiments, a non-limiting example of a delivery device is a syringe that includes at least a hollow barrel that forms an internal space, a plunger that is coupled and fitted into the hollow barrel, and a needle that is coupled to the barrel, the needle including a space that is contiguous with the internal space of the hollow barrel when the needle is coupled with the barrel. Both the plunger and the needle may be either directly or indirectly coupled to the hollow barrel. The syringe is constructed to deliver the PEVs and/or naked mitochondria intraocularly or intravitreally. In some embodiments, the syringe is constructed to deliver the PEVs and/or naked mitochondria subcutaneously. In some embodiments, the syringe is constructed to deliver the PEVs and/or naked mitochondria intravenously. In some embodiments, the syringe is constructed to deliver the PEVs and/or naked mitochondria at a subretinal location. In some embodiments, syringe is constructed to deliver the PEVs and/or naked mitochondria into the peritoneal cavity (intraperitoneal injection). In some embodiments, the syringe is constructed to deliver the PEVs and/or naked mitochondria systemically to the patient (enteric or parenteral).

In some embodiments, a non-limiting example of a delivery device is a port delivery system, which provides sustained release of PEVs and/or naked mitochondria (any of which may be optionally combined with other therapeutic agents) via intraocular or intravitreal delivery thereof. The port delivery system has been described by U.S. Pat. No. 9,968,603, the disclosure of which is hereby incorporated by reference.

As but one non-limiting example in some embodiments, Gyroscope Therapeutics, has developed the ORBIT™ subretinal delivery system that can be adapted for delivery of PEVs. Another delivery system involves providing the PEVs on contact-lenses, which are then placed in the affected eye. The PEVs can also be embedded in a gel-like material and deployed in “microneedles” as described by Lee et al., Advanced Functional Materials, doi.org/10.1002/adfm.202000086 (2020), the disclosure of which is hereby incorporated by reference. Many other such delivery systems are known and can be adapted to deliver PEVs.

In some embodiments, the blood is derived from a mammalian subject. According to another embodiment, the mammalian subject is a human subject. According to another embodiment, the mammalian subject is selected from a group consisting of: a human, a horse, a dog, a cat, a mouse, a rat, a cow, and a sheep. Each possibility represents a separate embodiment of the present invention. According to another embodiment, the PEVs of the invention are derived from a mammalian cell. According to another embodiment, the mammalian cell is a human cell. According to another embodiment, the PEVs are derived from cells in culture. According to another embodiment, the PEVs are derived from a tissue.

According to another embodiment, the PEVs are derived from a cell or a tissue selected from the group consisting of: human placenta, human placental cells grown in culture, and human blood cells. According to another embodiment, the PEVs of the invention are derived from a cell or a tissue selected from the group consisting of: placenta, hepatocytes, placental cells grown in culture, and blood cells. According to another embodiment, naked mitochondria may be isolated from a cell or a tissue selected from the group consisting of: liver, bone marrow, placenta, human placental cells, or any other tissues of a donor. According to another embodiment, naked mitochondria may be isolated from a cell grown in culture or a tissue grown in culture selected from the group consisting of: liver, bone marrow, placenta, human placental cells, or any other tissues of a donor.

As used herein, the phrase “naked mitochondria” refers to mitochondria that are isolated from the cell or the tissue. In some embodiments the cell is a cell grown in culture. In some embodiments, the tissue is tissue grown in culture. The naked mitochondria can be suspended in a freezing buffer, a hydrogel, a pharmaceutically acceptable liquid medium capable of supporting of the naked mitochondria, or a buffer solution which includes a saccharide. In some embodiments, the hydrogel is biocompatible, biodegradable, and capable of supporting naked mitochondria. In some embodiments, the hydrogel may be thermosensitive, which includes temperature-dependent hydrophilicity and hydrophobicity. In some embodiments, the hydrogel is biocompatible, biodegradable, capable of supporting naked mitochondria, and thermosensitive, the latter of which includes the hydrogel having temperature-dependent hydrophilicity and hydrophobicity.

As used herein, the phrases “cells grown in culture” or “a tissue grown in culture” refers to a multitude of cells or a tissue, respectively, grown in a liquid, semi-solid or solid medium, outside of the organism from which the cells or tissue derive. According to some embodiments, cells grown in culture are cells grown in bioreactors. According to a non-limiting example, cells may be grown in a bioreactor, followed by isolation of PEVs from the cells. According to another non-limiting example, cells may be grown in a bioreactor, which is followed by isolation of the mitochondria from the cells. In some embodiments, the isolated mitochondria from the cells grown in a bioreactor is naked mitochondria. According to a non-limiting example, a tissue may be grown in a bioreactor followed by isolation of PEVs from the cells of the tissue.

In some embodiments, the blood is from mice. In some embodiments, mouse blood is used to test the feasibility of the method of extracting PEVs. In some embodiments, the blood is from human donors.

Once blood is obtained, anticoagulant and a buffer are added to prevent blood from becoming thick and solid. In some embodiments, the anticoagulant is ACD (20%). In some embodiments, the buffer is 40% Tyrode's buffer having a pH of about 6 to about 7, preferably pH 6.5.

After adding anticoagulant and a buffer to blood, the mixture is then separated into supernatant and platelet rich plasma (PRP). In some embodiments, the separating is by centrifuging. Plasma is the liquid portion of whole blood. It is composed largely of water and proteins, and it provides a medium for red blood cells, white blood cells and platelets to circulate through the body. Platelets are blood cells that cause blood clots and other necessary growth healing functions. After the centrifuging of the mixture, blood cells are formed a pellet that accumulates at the bottom of a tube. The pellet is referred to as platelet rich plasma (PRP), which contains concentrated platelets.

Buffers are then added to the collected PRP to resuspend the platelets. The platelets are then activated or stimulated. There are many ways to activate platelets. Any of a number of substances can be used for this purpose including carbon radioisotopes, prostaglandins, serotonin, adenosine triphosphate, collagen, l-lactate dehydrogenase, thrombin, magnesium, adenosine, calcium, and heat-aggregated antibodies. In some embodiments, platelets are activated by freeze-thaw cycles. As used herein, the term “freeze-thaw cycle” refers to freezing of the mitochondria of the invention to a temperature below 0° C., maintaining the mitochondria in a temperature below 0° C. for a defined period of time and thawing the mitochondria to room temperature or body temperature or any temperature above 0° C. The term “room temperature”, as used herein refers to a temperature of between 18° C. and 25° C. The term “body temperature”, as used herein, refers to a temperature of between 35.5° C. and 37.5° C., preferably 37° C.

In another embodiment, mitochondria that have undergone a freeze-thaw cycle were frozen at a temperature of at least −70° C. In another embodiment, the mitochondria that have undergone a freeze-thaw cycle were frozen at a temperature of at least −20° C. In another embodiment, the mitochondria that have undergone a freeze-thaw cycle were frozen at a temperature of at least −4° C. In another embodiment, the mitochondria that have undergone a freeze-thaw cycle were frozen at a temperature of at least 0° C. According to another embodiment, freezing of the mitochondria is gradual. According to some embodiment, freezing of mitochondria is through flash-freezing. As used herein, the term “flash-freezing” refers to rapidly freezing the mitochondria by subjecting them to cryogenic temperatures.

In another embodiment, the mitochondria that underwent a freeze-thaw cycle were frozen for at least 30 minutes prior to thawing. According to another embodiment, the freeze-thaw cycle comprises freezing the partially purified functional mitochondria for at least 30, 60, 90, 120, 180, 210 minutes prior to thawing. Each possibility represents a separate embodiment of the present invention. In another embodiment, the mitochondria that have undergone a freeze-thaw cycle were frozen for at least 1, 2, 3, 4, 5, 6, 7, 8, 9, 10, 24, 48, 72, 96, 120 hours prior to thawing. Each freezing time presents a separate embodiment of the present invention. In another embodiment, the mitochondria that have undergone a freeze-thaw cycle were frozen for at least 4, 5, 6, 7, 30, 60, 120, 365 days prior to thawing. Each freezing time presents a separate embodiment of the present invention. According to another embodiment, the freeze-thaw cycle comprises freezing the partially purified functional mitochondria for at least 1, 2, 3 weeks prior to thawing. Each possibility represents a separate embodiment of the present invention. According to another embodiment, the freeze-thaw cycle comprises freezing the partially purified functional mitochondria for at least 1, 2, 3, 4, 5, 6 months prior to thawing. Each possibility represents a separate embodiment of the present invention.

According to another embodiment, the mitochondria that underwent a freeze-thaw cycle were frozen within a freezing buffer. According to another embodiment, the mitochondria that underwent a freeze-thaw cycle were frozen within the isolation buffer. As used herein, the term “isolation buffer” refers to a buffer in which the mitochondria of the invention have been partially purified. In a non-limiting example, the isolation buffer comprises 200 mM sucrose, 10 mM Tris-MOPS and 1 mM EGTA. According to some embodiments, BSA (Bovine Serum Albumin) is added to the isolation buffer during partial purification. According to some embodiments, 0.2% BSA is added to the isolation buffer during partial purification. According to some embodiments, HSA (Human Serum Albumin) is added to the isolation buffer during partial purification. According to some embodiments, 0.2% HSA is added to the isolation buffer during partial purification. According to other embodiment, HSA or BSA is washed away from the mitochondria of the invention following partial purification. Each possibility represents a separate embodiment of the present invention. Without wishing to be bound by any mechanism or theory, freezing mitochondria within the isolation buffer saves time and isolation steps, as there is no need to replace the isolation buffer with a freezing buffer prior to freezing or to replace the freezing buffer upon thawing.

According to another embodiment, the freezing buffer comprises a cryoprotectant. According to some embodiments, the cryoprotectant is a saccharide, an oligosaccharide, or a polysaccharide. Each possibility represents a separate embodiment of the present invention. According to another embodiment, the saccharide concentration in the freezing buffer is a sufficient saccharide concentration which acts to preserve mitochondrial function. According to another embodiment, the isolation buffer comprises a saccharide. According to another embodiment, the saccharide concentration in the isolation buffer is a sufficient saccharide concentration which acts to preserve mitochondrial function. According to another embodiment, the saccharide is sucrose. According to another embodiment, the saccharide is other than trehalose. Without wishing to be bound by any theory or mechanism, mitochondria that have been frozen within a freezing buffer or isolation buffer comprising sucrose demonstrate a comparable or higher oxygen consumption rate following thawing, as compared to control mitochondria that have not undergone a freeze-thaw cycle or that have been frozen within a freezing buffer or isolation buffer without sucrose.

According to some embodiments, addition of a saccharide to the mitochondria composition of the invention at a sufficient concentration acts to preserve mitochondrial function. According to another embodiment, a sufficient saccharide concentration which acts to preserve mitochondrial function is a concentration of between 100 mM-400 mM, preferably between 100 mM-250 mM, most preferably between 200 mM-250 mM. Each possibility represents a separate embodiment of the present invention. According to another embodiment, the saccharide according to the invention is sucrose. According to some embodiments the saccharide of the invention is other than trehalose. According to some embodiments the saccharide of the invention is other than mannitol.

According to another embodiment, the saccharide concentration in the composition of the invention is between 100 mM-150 mM. According to another embodiment, the saccharide concentration in the composition of the invention is between 150 mM-200 mM. According to another embodiment, the saccharide concentration in the composition of the invention is between 100 mM-200 mM. According to another embodiment, the saccharide concentration in the composition of the invention is between 100 mM-400 mM. According to another embodiment, the saccharide concentration in the composition of the invention is between 150 mM-400 mM. According to another embodiment, the saccharide concentration in the composition of the invention is between 200 mM-400 mM. According to another embodiment, the saccharide concentration in the composition of the invention is at least 100 mM. According to another embodiment, the saccharide concentration in the composition of the invention is at least 200 mM. Without wishing to be bound by any theory or mechanism of action, a saccharide concentration below 100 mM may not be sufficient to preserve mitochondrial function.

In some embodiments, the stimulant is heat aggregated-IgG.

Stimulated-platelets are centrifuged to remove remnant platelets or cells. The Supernatant containing the PEVs are then collected.

According to some embodiments, the PEVs are derived from the subject in need thereof. According to another embodiment, the PEVs are derived from a different subject than the subject in need thereof. According to another embodiment, the PEVs are derived from the same subject to whom they are administered. According to another embodiment, the PEVs are derived from a different subject than the subject to whom they are administered. According to another embodiment, the PEVs of the invention are from a source selected from autologous, allogeneic, and xenogeneic. Each possibility represents a separate embodiment of the present invention. As used herein, mitochondria of an autologous source refer to mitochondria derived from the same subject to be treated. As used herein, mitochondria of an allogeneic source refer to mitochondria derived from a different subject than the subject to be treated from the same species. As used herein, mitochondria of a xenogeneic source refer to mitochondria derived from a different subject than the subject to be treated from a different species. According to another embodiment, the PEVs of the invention are derived from a donor. According to some embodiments, the donor is an allogeneic donor. According to some embodiments, the donor is an autologous donor.

The term “subject in need thereof”, as used herein, refers to a subject afflicted with, or at a risk of being afflicted with, a condition which benefits from increased mitochondrial function. In some embodiments, the condition includes a retinal disease or condition. In some embodiments, the condition includes cardiogenic shock. In some embodiments, the condition includes sepsis. In some embodiments, the condition includes COVID-19. Each possibility represents a separate embodiment of the present invention. According to some embodiments, “a subject in need thereof” is a subject afflicted with a condition which may benefit from pro-apoptotic activity. In a non-limiting example, a condition which may benefit from pro-apoptotic activity is cancer. According to another embodiment, a subject in need thereof is mammalian. According to another embodiment, a subject in need thereof is human. According to another embodiment, a subject in need thereof is selected from the group consisting of: a human, a horse, a dog, a cat, a mouse, a rat, a cow and a sheep.

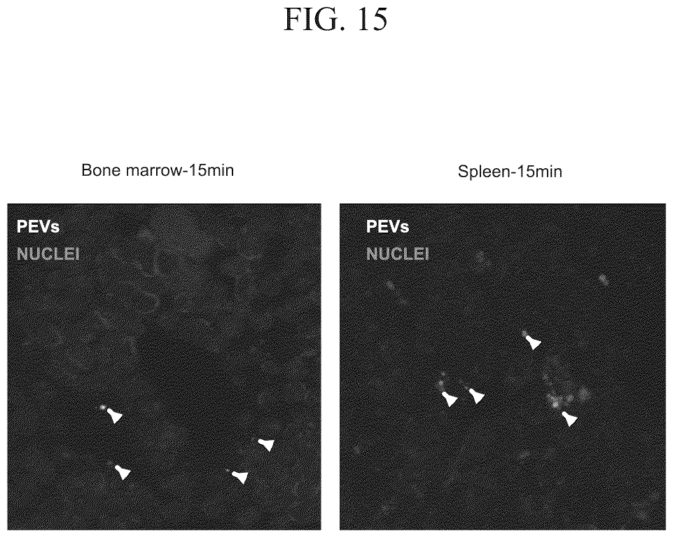

Some embodiments relate to a method of transducing platelet-derived mitochondria-containing extracellular vesicles (PEVs) into cells. The method includes 1) extracting PEVs from blood, and 2) incubating the PEVs with the cells for a time sufficient to transduce the PEVs into the cells. Incubating the cells can be in vitro or in vivo, the latter shown in FIG. 15.

In certain in vivo embodiments, incubating the PEVs with the cells comprises injecting the PEVs into blood, cerebrospinal fluid, pleural fluid, pericardial fluid, peritoneal/ascitic fluid, synovial fluid, saliva, or any other bodily fluid of a subject. This can be accomplished in a variety of manners, including use of an appropriate catheter, such as an intra-arterial or intrathecal catheter. The PEVs can also be introduced into a specific organ or tissue of a subject, such as an eyeball or retina of the subject. For this purpose, PEVs can be delivered via intra-vitreal, intravenous, or intra-arterial injections.

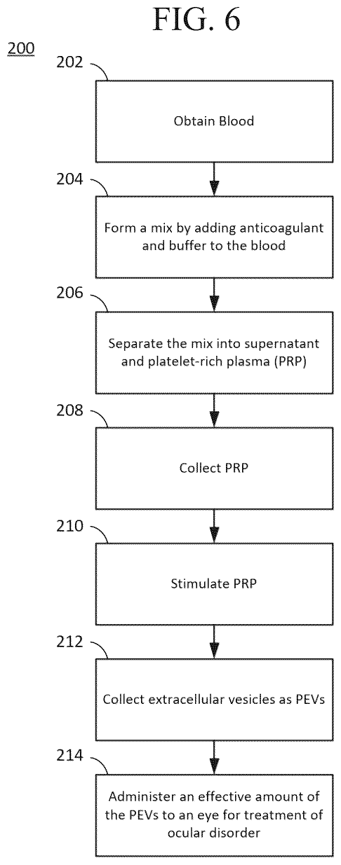

According to another embodiment, as shown in FIG. 6, a method 200 of treatment for ocular disorders, or a symptom of the ocular disorder, in a patient in need thereof is provided, the method comprising obtaining platelet-derived extracellular vesicles that include mitochondria (PEVs). The PEVs have been collected by: obtaining blood from one or more donors in step 202; adding an anticoagulant and a buffer to the blood to form a mix in step 204; separating the mix into supernatant and platelet rich plasma (PRP) in step 206; collecting the PRP in step 208; stimulating the collected PRP in step 210, thereby expelling extracellular vesicles from platelets in the PRP; and collecting the extracellular vesicles as PEVs in step 212, many of these steps related to the collecting of the PEVs have been described in other embodiments, and thus, are similar. In some embodiments, the method further comprises administering an effective amount of the PEVs to the eye of the patient in step 214, thereby treating the ocular disorder. In some embodiments, the PEVs have been collected at a different site than a site where the treatment is carried out. In some embodiments, the PEVs are collected on-site or off-site.

As used herein, the term “on-site” refers to a location at which the administration step is performed or is to be performed. The location can be in the same room, office, or ward that the administration step is performed or is to be performed. The location can be in a same building that the administration step is performed or is to be performed. The location can be in the same building complex that includes a plurality of buildings, at least one of the plurality of buildings is where the administration step is performed or is to be performed. The building complex can have the same affiliation (business or organization) or at least one of the plurality of the buildings may have a different affiliation.

As used herein, the term “off-site” refers to an outside location that is apart from the location at which the administration step is performed or is to be performed. The outside location can be a room or a laboratory that is apart from the building and the building complex (if the building is part of the building complex) where the administration step is performed or is to be performed.

In some embodiments, the blood has been stored for about four days or more. In some embodiments, the blood has been stored for about one year. In some embodiments, the PEVs are frozen while stored. In some embodiments, the frozen PEVs are stored in combination with a cryoprotectant. In some embodiments, the cryoprotectant is selected from the group consisting of a saccharide, an oligosaccharide, and a polysaccharide. In some embodiments, the anticoagulant is anticoagulant citrate dextrose (ACD). In some embodiments, the buffer is Tyrode's buffer at about pH 6 to about pH 7, preferably at about pH 6.5. In some embodiments, the separating is conducted by centrifuge. In some embodiments, for the stimulating step, the collected PEVs are stimulated with immune complexes in the presence of Ca²⁺. In some embodiments, the immune complexes comprise heat aggregated-IgG. The concentration of the heat-aggregated Ig used in the stimulation step is preferably about 0.1 mg/mL to about 2.5 mg/mL, more preferably about 0.5 mg/mL. The concentration of the Ca²⁺ used in the stimulation step is about 1 mM to about 25 mM, more preferably about 5 mM. In some embodiments, the collected PRPs are stimulated by freeze-thaw cycles.

In some embodiments, after the administering the PEVs into the eye, the PEVs contact at least one cell of the eye. In some embodiments, the PEVs are internalized into the cell following the PEVs contacting the cell. As used herein, the terms “contact” and “contacting” refers to a composition, which includes mitochondria, that is in sufficient proximity to the cell to trigger internalization of at least the mitochondria into the cell.

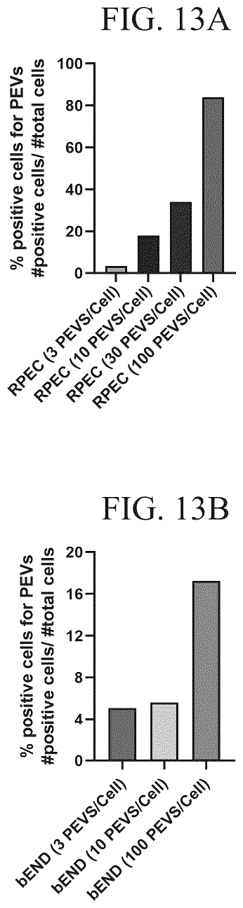

In some embodiments, the effective amount for treatment of the ocular disorder corresponds to an amount of the internalized PEVs, which ranges from about 3 PEV/cell to about 100 PEV/cell for at least one cell as shown for RPECs and bENDs in FIGS. 13A-B. In some embodiments, the effective amount corresponds to an amount of the internalized PEVs, which is about, for at least one cell, 3 PEV/cell, about 10 PEV/cell, about 30 PEV/cell or about 100 PEV/cell. The effective amount will vary, as recognized by those skilled in art, depending on the route of administration, possibility of co-administration with another therapeutic product(s), possibility of co-usage with another therapeutic treatment(s) or method(s), type(s) of delivery device(s) used, and usage of any excipients.

In some embodiments, the ocular disorder to be treated is aged macular degeneration (AMD). AMD is an ocular disorder that is one of the leading causes of vision loss, particularly in developed countries, having a prevalence of up to around 40%. AMD is characterized by mitochondrial dysfunction that affect the retina, this dysfunction brought upon by oxidative stress from reactive oxygen species (ROS). In AMD, ROS are produced at high levels in the RPE cells, which causes damage to mtDNA. The poor repair mechanisms of mtDNA allow this damage to accumulate over time to the point of causing the death of the mitochondria cells, which then leads to the death of the RPE cells. Because RPE cells support the health of photoreceptors, the death of the RPE cells lead to the demise of the photoreceptors that they support, which leads to visual loss.

In some embodiments, the ocular disorder to be treated is retinitis pigmentosa (RP). RP is an inherited disorder of the eye that causes severe vision impairment and is characterized by rod degeneration. In some embodiments, the ocular disorder is Leber's Hereditary Optical Neuropathy (LHON), a hereditary mitochondrial genetic disorder that is manifested by three primary mtDNA mutations in 90% of the cases. From these mtDNA mutations, LHON primarily affects retinal ganglion cells (RGC) causing their degeneration, which leads to vision loss. In some embodiments, the ocular disorder is diabetic retinopathy, which is characterized by the dysfunction of endothelial cells of the retinal microvasculature and the supporting cells of the retina such as Muller cells. In diabetic retinopathy, dysfunction of the endothelial cells leads to increased permeability thereof, which may bring about vascular leakage. This vascular leakage may cause edema in the surrounding, and thus, may lead to other relevant retinal diseases such as diabetic macular edema. In some embodiments, the ocular disorder is glaucoma.

In some embodiments, the cell to receive the PEVs includes a retinal pigment epithelium cell. In some embodiments, the cell to receive the PEVs includes a retinal ganglion cell. In some embodiments, the cell to receive the PEVs is located about a macula of the eye.

Specific tissues and organs can be specifically targeted by complexing the PEVs with specific receptors or coatings that facilitate “homing” to certain cell types. For example, U.S. Pat. No. 10,537,594, the contents of which are hereby incorporated by reference, exemplifies the use of asialoglycoprotein (AsG) receptor system to target mitochondria to liver cells. Similar systems can be used to target other tissues or organs.

Various techniques can be employed to facilitate internalization of mitochondria into cells both in vitro and in vivo. It is believed that high levels of mitochondrial internalization can be achieved for specific diseased tissues, or for the elderly, because in these cases the tissues are severely lacking in mitochondria. Cells that are energy-deficient will be expected to activate chemical pathways enabling easier internalization. When a free mitochondria or mitochondria-packed stem cell floats by, the cells signal their need and/or engulf the mitochondria. Even higher uptake of mitochondria can be obtained by adjusting timing, frequency, and duration so as to maintain large quantities of continuously in the bloodstream to ensure that there is always a ready supply. Precision placement can also be employed by injecting mitochondrial substance directly into an organ via an arterial shunt. For example, the hepatic artery can be used to concentrate mitochondria into the liver to preferentially regenerate liver tissue. By doing this properly, processes, such as clonal expansion, which threaten to dilute or reverse the mitochondria can be bypassed.

Diathermy and exercise by the subject can also facilitate uptake of mitochondria by cells. Exercise causes skeletal muscles to create more mitochondria. This is expected cause cells to accept more transplants. Research indicates this effect might be triggered also by heating the muscle with RF radio energy or ultrasound, for 2-4 hours per session.

Caloric restrictions/fasting by the subject can also facilitate uptake of mitochondria. Studies show that fasting causes mitochondrial changes, fission/fusion, or mitophagy. Perhaps some sequencing of fasts with transfusions would trick the cells into accepting a few extra mitochondria and more quickly share the new mtDNA with the mitochondrial network.

Other techniques can also be employed to facilitate to uptake of mitochondria. For example, partial poisoning, chemotherapy, or hypoxia to induce autophagy, followed by transfusion, repeated many times can be employed. In addition, metformin, melatonin, or other drugs are believed to stimulate uptake of mitochondria by cells. The use of drugs to shut down mitochondria's ability to reproduce itself by cloning can force the cells to rely only on transfused mitochondria.

Some embodiments relate to a method of increasing respiration of cells. The method includes transducing the cells with isolated PEVs according to the method described herein and producing ATP from the PEVs.

The isolated PEVs include functional mitochondria. In some embodiments, the term “functional mitochondria” refers to mitochondria that consume oxygen. In another embodiment, functional mitochondria have an intact outer membrane. Other embodiments include mitochondrial fragments, mitochondrial DNA, or segments thereof and mRNAs encoding mitochondrial gene products. In some embodiments, functional mitochondria are intact mitochondria. In another embodiment, functional mitochondria consume oxygen at an increasing rate over time. In another embodiment, the functionality of mitochondria is measured by oxygen consumption. In another embodiment, oxygen consumption of mitochondria may be measured by any method known in the art. According to some embodiments, functional mitochondria are mitochondria which display an increase in the rate of oxygen consumption in the presence of ADP and a substrate such as, but not limited to, glutamate, malate, or succinate. Each possibility represents a separate embodiment of the present invention. In another embodiment, functional mitochondria are mitochondria which produce ATP. In another embodiment, functional mitochondria are mitochondria capable of manufacturing their own RNAs and proteins and are self-reproducing structures. In another embodiment, functional mitochondria produce a mitochondrial ribosome and mitochondrial tRNA molecules.

As used herein, the term “intact mitochondria” refers to mitochondria comprising an outer and an inner membrane, an inter-membrane space, the cristae (formed by the inner membrane) and the matrix. In another embodiment, intact mitochondria comprise mitochondrial DNA. In another embodiment, intact mitochondria contain active respiratory chain complexes I-V embedded in the inner membrane. In another embodiment, intact mitochondria consume oxygen.

According to another embodiment, intactness of a mitochondrial membrane may be determined by any method known in the art. In a non-limiting example, intactness of a mitochondrial membrane is measured using the tetramethylrhodamine methyl ester (TMRM) or the tetramethylrhodamine ethyl ester (TMRE) fluorescent probes. Each possibility represents a separate embodiment of the present invention. Mitochondria that were observed under a microscope and show TMRM or TMRE staining have an intact mitochondrial outer membrane.

As used herein, the term “a mitochondrial membrane” refers to a mitochondrial membrane selected from the group consisting of: the mitochondrial inner membrane, the mitochondrial outer membrane, or a combination thereof.

In some embodiments, the functional mitochondria are partially purified mitochondria. As used herein, the term “partially purified mitochondria” refers to mitochondria separated from other cellular components, wherein the weight of the mitochondria constitutes between 20-80%, preferably 30-80%, most preferably 40-70% of the combined weight of the mitochondria and other sub-cellular fractions (as exemplified in: Hartwig et al., Proteomics, 2009, (9):3209-3214), the disclosure of which is hereby incorporated by reference. Each possibility represents a separate embodiment of the present invention.

According to another embodiment, partially purified mitochondria do not contain intact cells. According to another embodiment, the composition of the invention does not comprise intact cells. According to another embodiment, the composition of the invention does not comprise mitochondrial clumps or aggregates or cellular debris or components larger than 5 m. Each possibility represents a separate embodiment of the present invention. According to another embodiment, the composition of the invention is devoid of particulate matter greater than 5 m. As used herein, the term “particulate matter” refers to intact cells, cell debris, aggregates of mitochondria, aggregates of cellular debris or a combination thereof. Each possibility represents a separate embodiment of the present invention. As used herein, a composition devoid of exogenous particulate matter greater than 5 m comprises no more than 1 M of particulate matter greater than 5 m, preferably less than 0.5 μM, most preferably less than 0.1 μM.

According to some embodiments, intact cells, cell debris or aggregates are removed from the composition of the invention. According to some embodiments, the composition of the invention is filtered through a filter of no more than 5 m, in order to remove any intact cells, cell debris or aggregates, as exemplified herein below. Without wishing to be bound by any theory or mechanism, use of compositions comprising mitochondrial clumps according to the methods of the invention may be less efficient and even detrimental to the subject. According to another embodiment, the composition of the invention does not comprise liposomes or any other particulate carrier. Each possibility represents a separate embodiment of the present invention.

According to another embodiment, the weight of the mitochondria in partially purified mitochondria constitutes at least 20% of the combined weight of the mitochondria and other sub-cellular fractions. According to another embodiment, the weight of the mitochondria in partially purified mitochondria constitutes between 20%-40% of the combined weight of the mitochondria and other sub-cellular fractions. According to another embodiment, the weight of the mitochondria in partially purified mitochondria constitutes between 40%-80% of the combined weight of the mitochondria and other sub-cellular fractions. According to another embodiment, the weight of the mitochondria in partially purified mitochondria constitutes between 30%-70% of the combined weight of the mitochondria and other sub-cellular fractions. According to another embodiment, the weight of the mitochondria in partially purified mitochondria constitutes between 50%-70% of the combined weight of the mitochondria and other sub-cellular fractions. According to another embodiment, the weight of the mitochondria in partially purified mitochondria constitutes between 60%-70% of the combined weight of the mitochondria and other sub-cellular fractions. According to another embodiment, the weight of the mitochondria in partially purified mitochondria constitutes less than 80% of the combined weight of the mitochondria and other sub-cellular fractions.

According to another embodiment, a method of treatment is provided for a condition, or symptoms thereof, in a subject having the condition, comprising obtaining mitlets comprising platelet-derived extracellular vesicles (PEVs), which include mitochondria; and administering an effective amount of the mitlets into the subject, thereby treating the condition, or the symptoms thereof. In some embodiments, the condition includes cardiogenic shock. In some embodiments, the condition includes sepsis. In some embodiments, the condition comprises a disease caused by a virus. In some embodiments, the virus comprises a coronavirus. In some embodiments, the coronavirus comprises SARS-CoV-2. In some embodiments, the disease is COVID-19. In some embodiments, the condition comprises cardiogenic shock, sepsis, and a disease caused by a virus. In some embodiments, the disease is COVID-19. In some embodiments, the disease is Long COVID-19. In some embodiments, the COVID-19 precedes the cardiogenic shock, and wherein the cardiogenic shock precedes the sepsis.

As in other embodiments, the PEVs are collected by obtaining blood from one or more donors; adding an anticoagulant and a buffer to the blood to form a mix; separating the mix into supernatant and platelet rich plasma (PRP); collecting the PRP; stimulating the collected PRP, thereby expelling extracellular vesicles from platelets in the PRP; and collecting the extracellular vesicles as the PEVs. In some embodiments, the PEVs have been collected at a different site than a site where the treatment is carried out.

In some embodiments, the administering step comprises injecting the effective amount of mitlets into the subject to treat the condition. In some embodiments, the injecting step comprises a local injection. In some embodiments, the local injection comprises an intracardiac injection. In some embodiments, the injecting step comprises an injection via an intramyocardial injection catheter. In some embodiments, the injecting step comprises a systemic injection. In some embodiments, the injection comprises an enteric injection. In some embodiments, the injecting step comprises parenteral injection. In some embodiments, the injecting step comprises an intravenous injection.

As in other embodiments, the collected PRP is stimulated with immune complexes in presence of Ca2+. In some embodiments, the immune complexes comprise heat-aggregated IgG. In some embodiments, the collected PRP is stimulated by freeze-thaw cycles. In some embodiments, the concentration of the heat-aggregated IgG is about 0.1 mg/mL to about 2.5 mg/mL, and wherein concentration of the Ca2+ is about 1 mM to about 25 mM. In some embodiments, the anticoagulant is anticoagulant citrate dextrose (ACD). In some embodiments, the buffer is Tyrode's buffer at about pH 6 to about pH 7. In some embodiments, the separating step is conducted by centrifuge.

In some embodiments, the blood has been stored for four or more days. In some embodiments, the blood has been stored for up to one year. In some embodiments, the mitlets are frozen while stored. In some embodiments, the frozen mitlets are stored in combination with a cryoprotectant. In some embodiments, the cryoprotectant is selected from the group consisting of a saccharide, an oligosaccharide, and a polysaccharide.

In some embodiments, during and/or after the administering the mitlets into the subject, the mitlets contact at least one cell of the subject. In some embodiments, the mitlets are internalized into the cell after the mitlets contact the cell. In some embodiments, the effective amount corresponds to an amount of the internalized mitlets, which ranges from about 3 mitlets/cell to about 100 mitlets/cell.

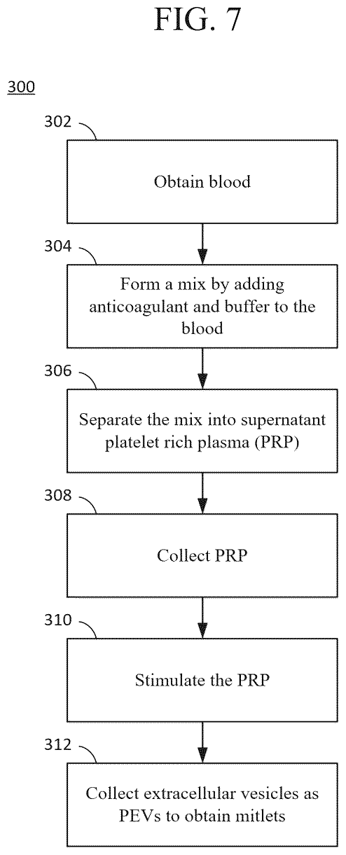

According to another embodiment, as shown in FIG. 7, a method 300 of treatment is provided for a condition, or symptoms thereof, in a subject having the condition, comprising obtaining PEVs from a source in step 302, wherein the PEVs comprise mitochondria; suspending the PEVs in a buffer to preserve the PEVs in step 308; and administering an effective amount of the PEVs into the subject in step 312, thereby treating the condition, or the symptoms thereof. In some embodiments, the source obtained in step 302 comprises a cell selected from the group consisting of: placental stem cells, umbilical cord stem cells, adipose tissue-derived stem cells; hepatocytes, blood cells, stem cells, or any cells from a donor. In some embodiments, the source comprises a tissue selected from the group consisting of: liver, bone marrow, placenta, adipose tissue, or any tissues from a donor.

In some embodiments, the obtaining step comprises growing the source in a bioreactor in step 304; and isolating the PEVs from the source grown in the bioreactor in step 306. In some embodiments, the obtaining step further comprise coating the PEVs in step 310 after isolating step 306. In some embodiments, the PEVs have been collected at a different site than a site where the treatment is carried out.

In some embodiments, the condition comprises cardiogenic shock. In some embodiments, the condition comprises sepsis. In some embodiments, the condition comprises a disease caused by a virus. In some embodiments, the virus comprises a coronavirus. In some embodiments, the coronavirus comprises SARS-CoV-2. In some embodiments, the disease is COVID-19. In some embodiments, the disease is Long COVID-19. In some embodiments, the condition comprises cardiogenic shock, sepsis, and a disease caused by a virus. In some embodiments, the disease is COVID-19. In some embodiments, the COVID-19 precedes the cardiogenic shock, and the cardiogenic shock precedes the sepsis. In some embodiments, the administering step comprises injecting the effective amount of mitlets into the subject to treat the condition.

In some embodiments, the buffer comprises a cryoprotectant. In some embodiments, the cryoprotectant is selected from the group consisting of a saccharide, an oligosaccharide, and a polysaccharide. In some embodiments, the buffer comprises a hydrogel. In some embodiments, the hydrogel has temperature-dependent hydrophilicity and hydrophobicity.

In some embodiments, mitlets are provided, the mitlets including platelet-derived extracellular vesicles (PEVs) that include mitochondria for use in the treatment of cardiogenic shock and/or sepsis, or symptoms thereof, wherein the PEVs are collected by: obtaining blood from one or more donors; adding an anticoagulant and a buffer to the blood to form a mix; separating the mix into supernatant and platelet rich plasma (PRP); collecting the PRP; stimulating the collected PRP, thereby expelling extracellular vesicles from platelets in the PRP; and collecting the extracellular vesicles as the PEVs. In some embodiments, the PEVs have been collected at a different site than a site where the treatment is carried out. In some embodiments, the mitlets are used in the treatment of cardiogenic shock. In some embodiments, the mitlets are used in the treatment of sepsis. In some embodiments, the cardiogenic shock and/or sepsis is caused by a virus. In some embodiments, the virus comprises a coronavirus. In some embodiments, the coronavirus comprises severe acute respiratory coronavirus 2 (SARS-CoV-2). In some embodiments, use in simultaneous treatment of cardiogenic shock, sepsis, and a disease caused by a virus. In some embodiments, the disease is COVID-19.

In some embodiments, the collected PRP is stimulated with immune complexes in presence of Ca2+. In some embodiments, the immune complexes comprise heat-aggregated IgG. In some embodiments, the collected PRP is stimulated by freeze-thaw cycles. In some embodiments, a concentration of the heat-aggregated IgG is about 0.1 mg/mL to about 2.5 mg/mL, and wherein concentration of the Ca2+ is about 1 mM to about 25 mM. In some embodiments, the anticoagulant is anticoagulant citrate dextrose (ACD). In some embodiments, the buffer is Tyrode's buffer at about pH 6 to about pH 7. In some embodiments, the separating step is conducted by centrifuge.

In some embodiments, the blood has been stored for four or more days. In some embodiments, the blood has been stored for up to one year. In some embodiments, an effective amount of the mitlets ranges from about 3 mitlets/cell to about 100 mitlets/cell. In some embodiments, the mitlets are frozen while stored. In some embodiments, the frozen mitlets are stored in combination with a cryoprotectant. In some embodiments, the cryoprotectant is selected from the group consisting of a saccharide, an oligosaccharide, and a polysaccharide.

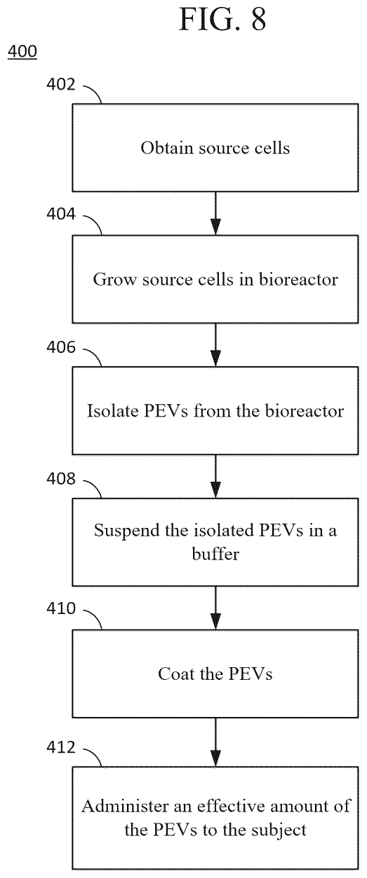

In some embodiments, PEVs are provided as shown in FIG. 8, the PEVs comprising mitochondria for use in the treatment of cardiogenic shock and/or sepsis, or symptoms thereof, wherein the PEVs are suspended in a buffer in step 408 to preserve the PEVs and are isolated in step 406 from source cells grown in a bioreactor in step 404, wherein the source cells obtained in step 402 are selected from the group consisting of: placental stem cells, umbilical cord stem cells, adipose tissue-derived stem cells; hepatocytes, blood cells, bone marrow, and induced pluripotent stem cells. In some embodiments, the PEVs are coated in step 410. In some embodiments, an effective amount of the PEVs are administered into a subject as shown in step 412. In some embodiments, the PEVs are used in the treatment of cardiogenic shock. In some embodiments, the PEVs are used in the treatment of sepsis. In some embodiments, the cardiogenic shock and/or sepsis is caused by a virus. In some embodiments, the virus includes a coronavirus. In some embodiments, the coronavirus includes severe acute respiratory coronavirus 2 (SARS-CoV-2). In some embodiments, the PEVs are used in simultaneous treatment of cardiogenic shock, sepsis, and a disease caused by a virus.

In some embodiments, the disease is COVID-19. In some embodiments, the buffer comprises a cryoprotectant. In some embodiments, the cryoprotectant is selected from the group consisting of a saccharide, an oligosaccharide, and a polysaccharide. In some embodiments, the buffer comprises a hydrogel. In some embodiments, the hydrogel has temperature-dependent hydrophilicity and hydrophobicity.

The following examples are presented to provide a more complete understanding of the invention. The specific techniques, conditions, materials, proportions and reported data set forth to illustrate the principles of the invention are exemplary and should not be construed as limiting the scope of the invention.

### Example 1

This example illustrates that PEVs are collected from mouse blood according to some embodiments.

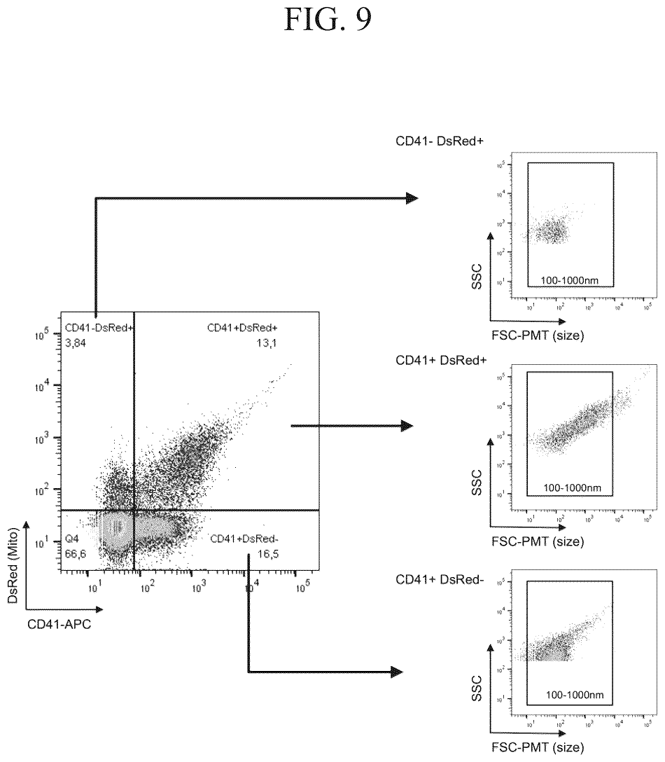

The following steps were conducted:
1. Blood was collected by from mouse donors. In this specific protocol, the donors were male DsRed mice, which are transgenic mice that express the red fluorescent protein variant DsRed. MST under the control of the chicken beta actin promoter coupled with the cytomegalovirus (CMV) immediate early enhancer. Here, 3×1 mL (1 mL/mouse) of blood was used.
2. ACD (20%) was added as an anticoagulant and 40% Tyrode's buffer pH 6.5 was also added to the blood. The blood mixture (20% ACD+40% Tyrode's buffer (TB) pH 6.5) was then centrifuged for 3 min at 500 g. PRP and buffy coat then collected and centrifuged for 2 min at 300 g.
3. PRP was collected and 20% ACD+10 mM EDTA added before a centrifugation step of 5 min at 1 300 g.
4. Each pellet was suspended in 0.1 mL TB pH 6.5 and 0.9 mL of TB pH 7.4 was added.
5. Platelets were pooled and counted using a cellometer and diluted at 10e8/mL in TB 7.4.
6. 900 million platelets were obtained in total and 5 mM CaCl₂) was added prior stimulation.
7. Platelets were stimulated overnight (16 h) at room temperature with heat aggregated-IgG at 0.5 mg/mL. Heat aggregated-IgG was prepared by aggregating human IgG (25 mg/mL, MPBIO) at 62° C. for 1 hour.
8. 10 mM EDTA was added to stop the stimulation.
9. Stimulated-platelets were centrifuged at 300 g for 5 min to remove remnant platelets or cells.
10. Supernatant was collected and PEVs were analyzed using a flow cytometer.
11. Remaining platelets were evaluated and represented less than 1% contamination.
12. Obtained PEVs were diluted 3 times with PBS and centrifuged at 18 000 g for 90 minutes at 18° C.
13. Pellet was resuspended in 0.3 mL PBS and PEVs counted by flow cytometry. Concentration was estimated at 1.5×10e9 PEVs/ml.
14. PEVs may be tagged with CD41 tags to enable them to be counted in a flow cytometer. If so tagged, PEVs represented approximatively 40% of the total CD41+PEVs. Dotplot representing PEV populations are illustrated in FIG. 9. (DsRed=PEVs).

### Example 2

This example illustrates that PEVs can be internalized by retinal cells according to some embodiments.
1. Immortalized mouse retinal pigmented epithelial cells (RPEC) and brain endothelial cells (bEND) were plated overnight prior to PEV incubation, approximately 20,000 cells/well (RPEC) and 16,000 cells/well (bEND).
2. PEVs were collected from mouse donors following the steps outlined in Example 1.
3. RPECs were preincubated with or without PEVs (about 3, 10, 30, or 100 mitochondria+PEVs per cell) for either 3, 18, 24, or 36 hours in Prigrow III, supplemented with 1% Pen-Strep (pH 7.4) and 1%, 5%, or 10% FBS (non-heat activated).
4. bENDs were preincubated with or without PEVs (about 3, 10, 30, or 100 mitochondria+PEVs per cell) for 24 hours in DMEM, supplemented with 1% Pen-Strep (pH 7.4) and 1%, 5%, or 10% FBS (non-heat activated).
5. RPECs and bENDs were washed and put in XF medium supplemented with 2 mM glutamine, 1 mM pyruvate, and 8 mM D-glucose and 1% FBS at a pH of 7.4.
6. After the wash, the RPECs and bENDs in XF medium were centrifuged at 300 g for 5 min and 60 mins at 37° C. without CO₂.

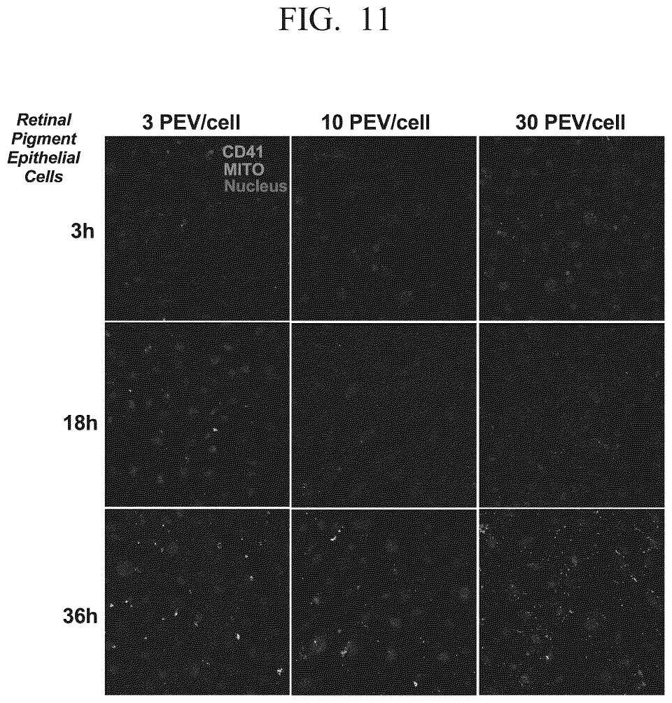

FIGS. 10A-B are confocal images that show stained nuclei 502 [DAPI (4′,6-diamidino-2-phenylindole)] and cellular membranes 504 of RPECs 500, and PEVs 510 (DsRed). As shown in FIGS. 10A-B (40× and 20× magnification respectively), the PEVs 510 are largely internalized by the RPECs 500. As shown in FIG. 11, this internalization may be stable well over 24 hours.

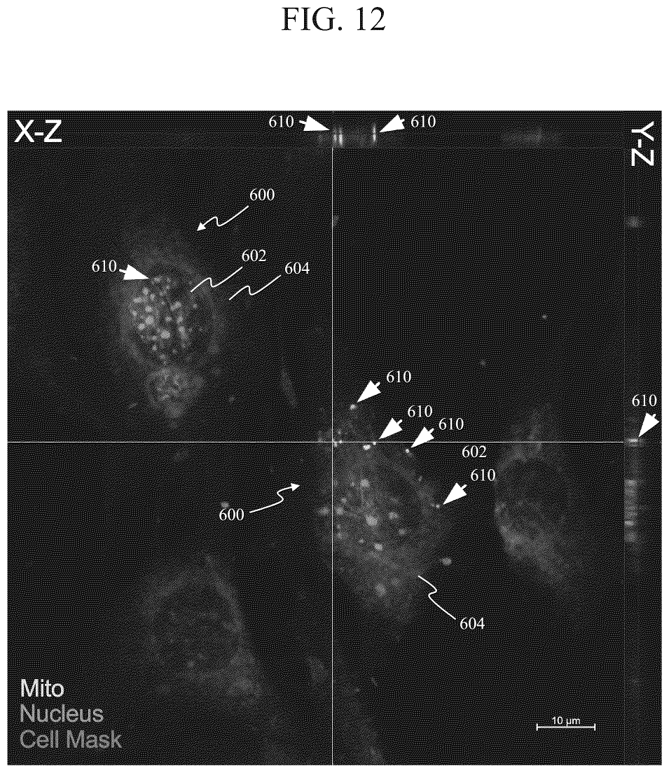

To verify mitochondria internalization in RPECs 600, X-Z and Y-Z scans of fluorescent-labeled mitochondria 610 (here, represented in orange) from PEVs were performed using a confocal microscope as shown in FIG. 12. Here, the X-Z plane is perpendicular to the Y-Z plane. The X-Z, Y-Z scans show how the highest intensity from the point source of the fluorescently labeled mitochondria (see arrowheads) is located within the RPEC 600. The nucleus 502 of the RPECs 600 were stained with DAPI. The cell membrane 604 of the RPECs 600 were stained with CellMask™ as indicated in FIG. 12. The results of this internalization are shown in FIG. 13A. FIG. 13B shows that bENDs also internalized mitochondria that were delivered by PEVs. Accordingly, these results demonstrate that PEVs can deliver durable mitochondria into RPECs and bENDs.

To assess the activity of the mitochondria delivered by PEVs, a Seahorse XF Assay that tests for mitochondria stress was performed on the RPECs that were preincubated with PEVs for 24 hours. The oxygen consumption rate (OCR) was measured between different preincubation parameters for RPEC (0, 3, 10, 30, or 100 PEVs/cell) as provided in step 3 of this example with 5% FBS appearing to be the preferred level of serum used; higher serum levels like 10% FBS may trigger cellular division.

With respect to the OCR readings of FIGS. 14A-14D, similar (if not slightly lower) basal respiration levels between the RPECs that were preincubated with PEVs and those that were not (controls), are shown in FIG. 14A. Basal respiration levels refer to the energetic demand of the RPECs under baseline conditions. Increased spare respiratory capacity of RPECs that were preincubated with PEVs is shown in FIG. 14B, the results of which indicate improvement in the capability of the RPEC to respond to an energic demand (i.e., improved cell fitness or flexibility). FIG. 14C appears to show slightly enhanced ATP production in some of the RPECs that were preincubated with PEVs. FIG. 14D shows that proton leak does not appear to be an issue with RPECs that internalized PEVs compared to those that did not.

### Example 3

A protocol for a sepsis pre-clinical study is summarized in Table 1 below. The subjects herein for this Example are mice. Sepsis is induced in the abdomen of mice not belonging to the sham group. Treatment groups 1 and 2 will receive a 120 L infusion of mitlets via intravenous injection (IV) at 1× and 5× dose respectively. Treatment groups 3 and 4 will receive a 120 L infusion of isolated mitochondria from liver tissue via IV at 1 Y and 5 Y respectively. The dose of X and Y in these experiments can be the same or different. Positive and negative controls will receive an infusion of an antibiotic cocktail and saline respectively. The mice in the sham group will not be induced with sepsis nor will receive any reagent.

The frequency of IV infusions will be daily for three consecutive days (12 hours post-induction of sepsis, 1 d, and 2 d). End points include a cytokine panel that measures cytokine levels over time for each group, a mortality graph for each group over time, and a muscle weakness chart. Endpoint readouts include baseline (0 d, 12 hours post-induction of sepsis, 1 d, 3 d, 7 d, 10 d, and 14 d).

TABLE 1

| Treatment Group | Reagent | Dose | Route | # animals |
|---|---|---|---|---|
| Sham (no sepsis) | None | None | None | 5 |
| Negative control | Saline | volume | IV | 5 |
| Positive control | Antibiotic cocktail | standard | IV | 5 |
| Treatment group 1 | Mitlets | 1X | IV | 10 |
| Treatment group 2 | Mitlets | 5X | IV | 10 |
| Treatment group 3 | Liver mitochondria | 1Y | IV | 10 |
| Treatment group 4 | Liver mitochondria | 5Y | IV | 10 |

### Example 4

Patients greater than, or equal to, 18 years of age are included in the study based on several criteria. The included patients are diagnosed with acute ST elevation myocardial infarction (STEMI), which includes clinical presentation of elevated troponin and electrocardiographic abnormalities consistent with acute STEMI. Further, the included patients will exhibit a measured criteria indicative of cardiogenic shock, the criteria including the following: (1) SBP <90 mmHg, low SVC O₂ saturation (<70%); and (2) elevated lactate and pulmonary congestion or elevated CVP >12 mmHg. Additionally, the patients in the study will have moderate to severe left ventricular systolic dysfunction, defined as left ventricular ejection fraction (LVEF)<35% measured by echocardiography.

Those who will be excluded from the study includes those diagnosed with severe chronic obstructive pulmonary disease (COPD) having FEV1<1 L and FEV1/FVC <70% (FEV1: Forced expiratory volume in 1 s; FVC: Forced Vital Capacity). Other exclusion criteria include having a history of organ transplantation; active malignancy (except localized skin cancer); advanced cardiogenic shock state with multi-organ failure (persistently elevated lactate level >2, oliguria or anuria, mechanical ventilation, or elevated abnormal liver function tests (LFTs) >3× normal limit); and women of childbearing potential.

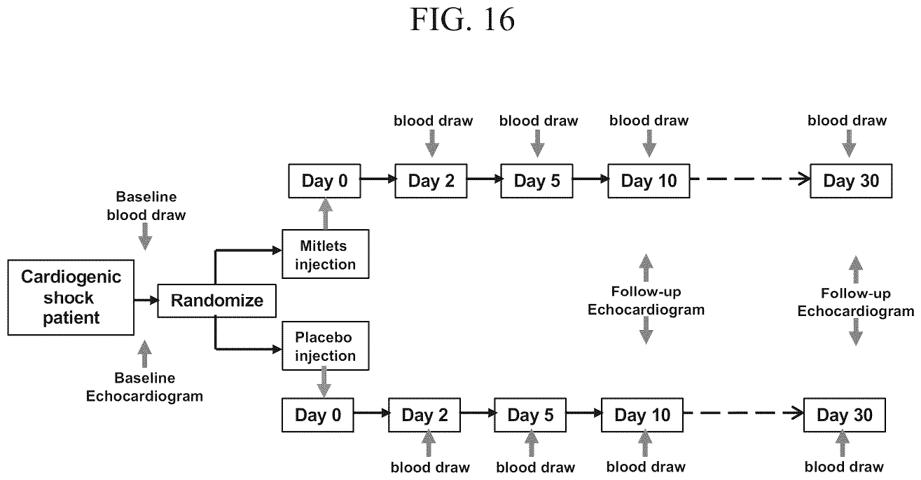

The study design is shown in FIG. 16, wherein the included patient's baseline echocardiogram (ECG) is measured, and blood is drawn for initial measurements. These patients will be randomized to receive either an injection of mitlets or placebo. The administration of the injection can be local or systemic. Blood draws at 2 d, 5 d, 10 d, and 30 d following the injection are used to measure the intensity of the inflammatory response associated with cardiogenic shock. Follow-up ECG's on 10 d and 30 d are performed. Markers, such as IL-1 (superfamily), IL-6, IL-8, TNF-α, c-reactive protein (CRP), soluble adhesion molecules, complement system, and others can be used to identify the inflammatory response.

The primary endpoints include changes in the inflammatory cytokine panel from baseline through the initial 10 d post-infusion of mitlets and any adverse effects. Secondary endpoints include survival at discharge; survival at 90 d; change in left ventricular function at 10 d (or hospital discharge if discharged prior to 10 d) and at 30 d; and any signs of major adverse cardiac events (MACE).

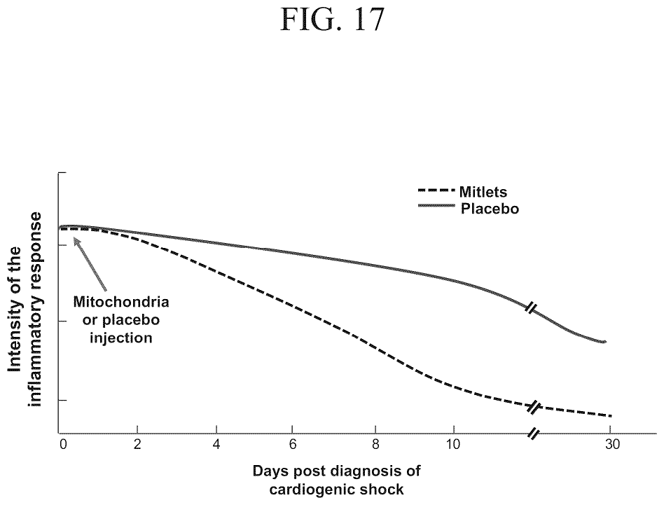

FIG. 17 measures the intensity of the inflammatory response based on the measured cytokine panel between the randomized group that are administered the mitlets and the randomized group that are administered with placebo. In this Example, administration route of the mitlets and the placebo were the same, which was IV. Although IV administration was used in this Example, other routes of administration are also possible, including intra-arterial, intraspinal, intraventricular, intraperitoneal, and intraosseous among others. As shown, the mitlet group displays a dramatic reduction in the intensity of the inflammatory response at 4 d post-diagnosis of cardiogenic shock compared to the placebo group. The difference in the intensity of the inflammatory response between the two groups further widens at 10 d post-diagnosis of the cardiogenic shock, which shows the intensity of the inflammatory response of mitlet group being >3× less than placebo group.

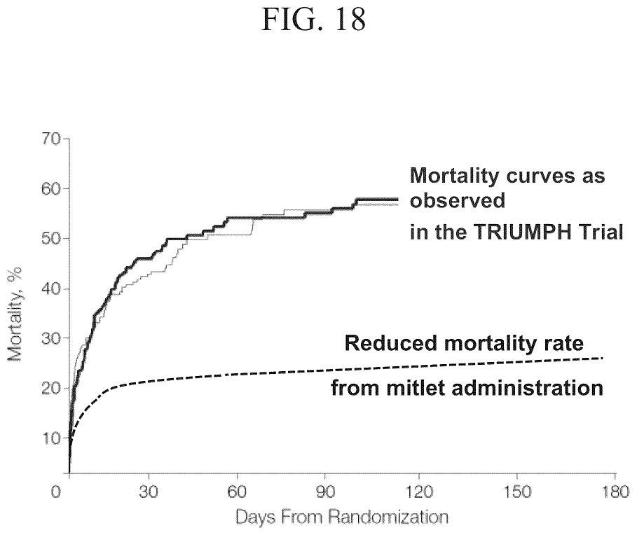

Accordingly, these results show the efficacy of an early and aggressive mitlet treatment in lowering the intensity of the inflammatory response from cardiogenic shock, and thus, the lower the probability of an onset of the cytokine storm. Therefore, the probability of sepsis and sepsis-related deaths from cardiogenic shock may be significantly reduced with early and aggressive clinical treatment based on mitlets as shown in FIG. 18.

The described embodiments and examples of the present disclosure are intended to be illustrative rather than restrictive, and are not intended to represent every embodiment or example of the present disclosure, and thus, are not to be limited in scope by the specific embodiments and examples described herein. While the fundamental novel features of the disclosure as applied to various specific embodiments thereof have been shown, described, and pointed out, it will also be understood that various omissions, substitutions, and changes in the details of the methods that are disclosed, may become apparent and may be made by those skilled in the art without departing from the spirit of the disclosure. For example, it is expressly intended that all combinations of those method steps that perform substantially the same function in substantially the same way to achieve the same results are within the scope of the disclosure. Moreover, it should be recognized that method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

What is claimed is:

1. A method of treatment of cardiogenic shock and/or sepsis, or symptoms thereof, in a subject having cardiogenic shock and/or sepsis, comprising:
obtaining mitlets comprising platelet-derived extracellular vesicles (PEVs) that include mitochondria, wherein the PEVs are collected by:
obtaining blood from one or more donors;
adding an anticoagulant and a buffer to the blood to form a mix;
separating the mix into supernatant and platelet rich plasma (PRP);
collecting the PRP;
stimulating the collected PRP, thereby expelling extracellular vesicles from platelets in the PRP; and
collecting the extracellular vesicles as the PEVs; and
administering an effective amount of the mitlets into the subject, thereby treating the cardiogenic shock and/or sepsis, or the symptoms thereof.

2. The method of claim 1, wherein the PEVs have been collected at a different site than a site where the treatment is carried out.

3. The method of claim 1, wherein the cardiogenic shock and/or sepsis is caused by a virus.

4. The method of claim 3, wherein the virus comprises a coronavirus.

5. The method of claim 4, wherein the coronavirus comprises severe acute respiratory coronavirus 2 (SARS-CoV-2).

6. The method of claim 1, wherein the subject has cardiogenic shock, sepsis, and a disease caused by a virus.

7. The method of claim 6, wherein the disease is COVID-19.

8. The method of claim 7, wherein the COVID-19 precedes the cardiogenic shock, and wherein the cardiogenic shock precedes the sepsis.

9. The method of claim 1, wherein the administering step comprises injecting the effective amount of mitlets into the subject to treat the condition.

10. The method of claim 1, wherein the collected PRP is stimulated with immune complexes in presence of Ca²⁺.

11. The method of claim 10, wherein the immune complexes comprise heat-aggregated IgG.

12. The method of claim 1, wherein the collected PRP is stimulated by freeze-thaw cycles.

13. The method of claim 11, wherein concentration of the heat-aggregated IgG is about 0.1 mg/mL to about 2.5 mg/mL, and wherein concentration of the Ca²⁺ is about 1 mM to about 25 mM.

14. The method of claim 1, wherein the anticoagulant is anticoagulant citrate dextrose (ACD).

15. The method of claim 1, wherein the buffer is Tyrode's buffer at about pH 6 to about pH 7.

16. The method of claim 1, wherein the separating step is conducted by centrifuge.

17. The method of claim 1, wherein the blood has been stored for four or more days.

18. The method of claim 1, wherein the effective amount corresponds to an amount of internalized mitlets, which ranges from about 3 mitlets/cell to about 100 mitlets/cell.

19. A method of treatment of cardiogenic shock and/or sepsis, or symptoms thereof, in a subject having the cardiogenic shock and/or sepsis, comprising:
administering an effective amount of PEVs containing mitochondria into the subject, thereby treating the condition, or the symptoms thereof, wherein the PEVs have been obtained by a method comprising:
obtaining source cells selected from the group consisting of: placental stem cells, umbilical cord stem cells, adipose tissue-derived stem cells; hepatocytes, blood cells, bone marrow, and induced pluripotent stem cells, wherein the PEVs comprise mitochondria;
growing the source cells in a bioreactor;
isolating the PEVs from the bioreactor; and
suspending the isolated PEVs in a buffer to preserve the PEVs.

20. A mitlet comprising platelet-derived extracellular vesicles (PEVs) that include mitochondria for use in the treatment of cardiogenic shock and/or sepsis, or symptoms thereof, wherein the PEVs are collected by:
obtaining blood from one or more donors;
adding an anticoagulant and a buffer to the blood to form a mix;
separating the mix into supernatant and platelet rich plasma (PRP);
collecting the PRP;
stimulating the collected PRP, thereby expelling extracellular vesicles from platelets in the PRP; and
collecting the extracellular vesicles as the PEVs.
